(19) United States
(12) Patent Application Publication
(10) Pub. No.: US 2018/0028842 A1
(43) Pub. Date: Feb. 1, 2018

(54) NAIL COATING HAVING MOSQUITO REPELLANT PROPERTIES

(71) Applicant: Jeff Pink, Beverly Hills, CA (US)

(72) Inventor: Jeff PINK, Beverly Hills, CA (US)

(21) Appl. No.: 15/662,736

(22) Filed: Jul. 28, 2017

Related U.S. Application Data
(60) Provisional application No. 62/367,719, filed on Jul. 28, 2016.

(51) Int. Cl. A61Q 3/02; A61K 8/34; A61K 8/42

(52) U.S. Cl. CPC A61Q 3/02; A61K 8/42; A61K 8/34

(57) ABSTRACT

The invention is a nail coating composition having slow release insect repellent properties, the at least one non-toxic active ingredient of the composition is selected from the group of consisting of naturally occurring plant extracts and essential oils, diethyl toluamide, ethyl hexanediol, and dimethyl phthalate.

### FIELD OF THE INVENTION

This invention relates to nail polish and top coats and in particular, a nail coating having insect repellent properties.

### BACKGROUND OF THE INVENTION

Nail polishes are ordinarily applied to human fingernails or toenails to decorate the nail. It is also known to apply a protective coating on top of the nail polish. Such compositions are often clear and are known as the “top coat” which protects the nail from chipping or breaking.

It is known to provide an adhesively applied strip to a fingernail, the strip containing a layer of DEET insect repellent encapsulated within a frangible shell layer of the strip. The frangible shell layer is manually ruptured by a user to selectively release the DEET repellent, the strip functioning as a barrier to prevent contact of the DEET repellent with the user's skin.

### BRIEF SUMMARY OF THE INVENTION

The present invention is a nail coating composition having slow release insect repellent properties. The composition is a liquid that is applied to the nail surface and following drying it slowly releases active ingredients having the insect repellant properties. Non-toxic active ingredient of the composition include a group of naturally occurring plant extracts and essential oils, N,N-Diethyl-m-Toluamide (DEET), 2-Ethyl-1,3-Hexanediol and Dimethyl Phthalate. In one preferred embodiment, an extract from the lemon eucalyptus plant or a synthetic version thereof, is provided within a lacquer based top coat composition as a first active in an amount of about 15% by total weight of the composition in combination with 15% by total weight of a second active ingredient that comprises a mixture of N,N-Diethyl-m-Toluamide (DEET), 2-Ethyl-1,3-Hexanediol and Dimethyl Phthalate.

### DETAILED DESCRIPTION OF THE INVENTION

In the present invention, the nail coating composition is preferably solvent based and will comprise a mixture of volatile solvents that, during mixing, evaporate away leaving the non-volatile ingredients behind, the remaining ingredients providing the nail coating with desired characteristics. Nail product solvents within the scope of the present invention are ethyl and butyl acetate. In addition, the coating composition will include one or more cellulose-based ingredients such as cellulose acetate butyrate to provide the correct shine, and various other ingredients known in the art, including but not limited to plasticizers, dyes, and the like to impart desired characteristics to the coating when applied to a nail. It is within the scope of the present invention to use non-polar organic solvents for the nail coating composition since some of the active ingredients within the scope of the invention are not water-soluble.

One active ingredient component of the present invention is selected from a group of plant based extracts having insect repellent characteristics, particularly mosquito repellency. For example, Corymbia citriodora (Myrtaceae), also known as lemon eucalyptus, contains a potent natural mosquito repellent that may be extracted from the leaves of lemon eucalyptus trees. The extract is known as para-menthane 3, 8 diol and it can be extracted from the eucalyptus leaves and twigs or be chemically synthesized. Another active ingredient component of the present invention comprises a mixture of N,N-Diethyl-m-Toluamide (DEET), 2-Ethyl-1,3-Hexanediol and Dimethyl Phthalate. It is within the scope of the present invention to combine these active ingredients within a single topcoat formula.

Other natural, plant based extracts having mosquito repellent characteristics that are within the scope of the present invention are essential oils extracted from citronella, cinnamon, thyme, catnip, clove and soybean, among others.

Table I is a solvent based clear top coat composition according to one embodiment of the present invention. The ingredients are in percentage by weight of the total composition.

TABLE I

| % | Ingredient |
|---|---|
| 48.039 | 20% of Eastman CAB 381.05 in a solution of 50:50 Ethyl |
| | and Butyl Acetate |
| 1.5 | 20% of Sucrose Benzoate (Genomer 6058) in a solution of |
| | Butyl Acetate |
| 1.95 | 50% Acrylate Copolymers in solution of Butyl Acetate, |
| | specifically, Pecorez AC-50 |
| 6.62 | Ethyl Acetate 99.5% |
| 4.31 | n-Butyl Acetate |
| 0.96 | Triphenyl Phosphate |
| 0.192 | Benzophenone-1 |
| 4.65 | SD Alcohol 40B 200 Proof |
| 1.51 | Trimethyl Pentanyl Diisobutyrate, specifically, |
| | Eastman TXIB |
| 0.268 | Dow Corning 200 Dimethicone |
| 0.001 | 1% Solution of Violet #2 Dye (Sensient K7014) in Butyl |
| | Acetate |
| 30.00 | p-Methane-3,8-diol, specifically, Coolact 38D |
| | (Takasago) |
| 100% | Total |

Preparing the composition in Table I is as follows. Explosion proof equipment should be used due to the extremely flammable nature of the volatile solvents used. Create a 20% solution of CAB in 80% of a mixture of 50:50 Ethyl:Butyl Acetate using a high speed disperser. Add this solution to the main kettle according to % weight. Create a 20% solution of Sucrose Benzoate in Butyl Acetate using a high speed disperser. Add this solution to the main kettle according to weight percentage. In a separate container dissolve the Triphenyl Phosphate and Benzophenone-1 in the Ethyl and Butyl Acetate and add this solution to the main kettle according to % weight. While mixing the product in the main kettle add the remaining ingredients one at a time by % weight and mix until clear and uniform.

The above composition is operable when using about 15 to about 30 weight percent of the p-Methane-3,8-diol active ingredient, with 30 weight percent being preferable.

Table II is a solvent based clear top coat composition according to another embodiment of the present invention. The ingredients are in percentage by weight of the total composition.

TABLE II

| % | Ingredient |
|---|---|
| 48.039 | 20% of Eastman CAB 381.05 in a solution of 50:50 Ethyl |
| | and Butyl Acetate |
| 1.5 | 20% of Sucrose Benzoate (Genomer 6058) in a solution |
| | of Butyl Acetate |
| 1.95 | 50% Acrylate Copolymers in solution of Butyl Acetate, |
| | specifically, Pecorez AC-50 |
| 6.62 | Ethyl Acetate 99.5% |
| 4.31 | n-Butyl Acetate 99.5% |
| 0.96 | Triphenyl Phosphate |
| 0.192 | Benzophenone-1 |
| 4.65 | SD Alcohol 40B 200 Proof |
| 1.51 | Trimethyl Pentanyl Diisobutyrate, specifically, |
| | Eastman TXIB |
| 0.268 | Dow Corning 200 Dimethicone |
| 0.001 | 1% Solution of Violet #2 Dye (Sensient K7014) in Butyl |
| | Acetate |
| 15.00 | UNIREP U-18 (N,N-Diethyl-m-Toluamide (DEET), 2-Ethyl- |
| | 1,3-Hexanediol and Dimethyl Phthalate) |
| 15.00 | Lemon of Oil Eucalyptus (Eucalyotus citriodora) |
| 100% | Total |

Preparing the composition in Table II is as follows. Explosion proof equipment should be used due to the extremely flammable nature of the volatile solvents used. Create a 20% solution of CAB in 80% of a mixture of 50:50 Ethyl:Butyl Acetate using a high speed disperser. Add this solution to the main kettle according to % weight. Create a 20% solution of Sucrose Benzoate in Butyl Acetate using a high speed disperser. Add this solution to the main kettle according to weight percentage. In a separate container dissolve the Triphenyl Phosphate and Benzophenone-1 in the Ethyl and Butyl Acetate and add this solution to the main kettle according to % weight. While mixing the product in the main kettle add the remaining ingredients one at a time by % weight and mix until clear and uniform.

UNIREP U-18 is manufactured and sold by Induchem AG of Switzerland. The Lemon of Eucalyptus oil ingredient is manufactured and sold by Down Under Enterprises International Pty LLd of Australia, the main constituent of which is citronellal (75-85%), with small amounts of citronellol, isopulegol and trace amounts of alpha-pinene, linalool, and bet-caryophyllene. Both components are the active ingredients within the Table II topcoat formula.

While this invention has been described as having a preferred design, it is understood that it is capable of further modifications, uses and adaptations, both in whole and in part, while following the general principle of the invention and including such departures from the present disclosure as is known or customary practice in the art to which this invention pertains, and as may be applied to the central features of this invention.

I claim:

1. A nail topcoat composition having sustained release insect repellant properties, the composition containing an effective amount of at least one active ingredient selected from the group consisting of lemon eucalyptus oil, diethyl toluamide, ethyl hexanediol, and dimethyl phthalate.
